(19) United States
(12) Patent Application Publication
(10) Pub. No.: US 2009/0111758 A1
(43) Pub. Date: Apr. 30, 2009

(54) USE OF THERAPEUTICALLY USEFUL PEPTIDES

(75) Inventors: Tiina Jauhiainen, Helsinki (FI); Riitta Korpela, Helsinki (FI); Heikki Vapaatalo, Tamper (FI); Mats Ronnback, Helsinki (FI)

Correspondence Address: NIXON & VANDERHYE, PC, 901 NORTH GLEBE ROAD, 11TH FLOOR, ARLINGTON, VA, 22203, US

(73) Assignee: VALIO LTD., HELSINKI (FI)

(21) Appl. No.: 12/300,901

(22) PCT Filed: May 15, 2006
(86) PCT No.: PCT/FI06/50194

§ 371 Date: Nov. 14, 2008

(51) Int. Cl. A61K 38/06

(52) U.S. Cl. 514/18

(57) ABSTRACT

The invention relates to preventing and treating endothelial dysfunction by using biologically active peptides and products containing them. In particular, the tripeptides Ile-Pro-Pro (IPP), Val-Pro-Pro (VPP) or mixtures, concentrates or other products containing them are used. A specific aspect of the present invention is to enhance the elasticity of blood vessels by using said biologically active peptides.

### FIELD OF THE INVENTION

The present invention relates to preventing and treating endothelial dysfunction by using biologically active peptides and products containing them. A product having a high short-chain peptide content has been found especially effective for use in accordance with the present invention.

The product to be used in accordance with the present invention can be formulated for instance as a health and wellness food product or a pharmaceutical product. It contains small-molecular peptides, such as the tripeptides Ile-Pro-Pro (IPP), Val-Pro-Pro (VPP), or mixtures or concentrates containing them, and it functions by improving epithelial function and curing diseases relating thereto. A specific aspect of the present invention is to reduce stiffness and thus enhance elasticity of blood vessels with the use of small-molecular peptides, such as tripeptides Ile-Pro-Pro (IPP), Val-Pro-Pro (VPP), or mixtures, concentrates and other products containing the same.

### BACKGROUND OF THE INVENTION

The vascular endothelium regulates locally vascular tone by the release of vasodilator substances, such as endothelium-derived relaxing factor (EDRF), nitric oxide, prostacyclin and endothelium-derived hyperpolarizing factor (EDHF) and vasoconstrictor substances, such as thromboxane A₂, free radicals and endothelin. The importance of nitric oxide in both basal and stimulated control of vascular tone in large epicardial coronary arteries and in the coronary microcirculation has been shown in several clinical studies (for review see Vapaatalo H, Mervaala E. Clinically important factors influencing endothelial function. Med Sci Monit 2001; 7(5):1075-1085. Drexler H, Hornig B. Endothelial dysfunction: a novel therapeutic target. J Mol Cell Cardiol 1999; 31:51-60). Above-mentioned different endothelial derived relaxing factors correct endothelial dysfunction e.g. in atherosclerosis. Regulation of vasomotor tone, modulation of blood coagulation, promotion and prevention of vascular growth, modulation of inflammation and action as a target to the effects and adverse effects of drugs are associated to endothelium. EDRF release is stimulated by increased flow in vessels of for example bradykinin, thrombin, acetylcholine and serotonin.

The endothelium controls underlying smooth muscle tone in response to certain pharmacological and physiological stimuli. The endothelial function plays also a role in vascular growth, leukocyte adhesion, and immunological regulation, metabolism of circulating amines, lipoprotein metabolism and integration and transduction of blood-borne signals.

Endothelial dysfunction is characterized by decreased secretion of vasodilatory mediators, increased production of vasoconstrictors, increased sensitivity to vasoconstrictors and/or resistance of vascular smooth muscle to endothelial vasodilators. Dysfunction is a consequence of an imbalance between relaxing and contracting factors, or growth promoting and inhibiting agents. Inflammation, lipoprotein oxidation or other oxidative stress reactions are factors affecting development and maintenance of endothelial dysfunction. Theoretically, the clearest and most direct indicators of endothelial dysfunction are nitric oxide and its metabolites, as well as cyclic GMP.

Endothelial dysfunction can be either a cause or a consequence of several clinical conditions, such as hypertension, atherosclerosis, coronary disease, heart failure, diabetes and high blood cholesterol. The most direct indicators of endothelial dysfunction are imbalance between decreased production or receptor function of vasodilatory factors, such as nitric oxide, prostacyclin, endothelium derived hyperpolarizing factor (EDHF), and natriuretic peptides, or increased formation of or sensitivity to vasoconstrictive agents, such as endothelin-1, angiotensin 11, endoperoxides, and thromboxane A₂.

Endothelial dysfunction can be treated with several known drugs, the most important being angiotensin converting enzyme (ACE) inhibitors, angiotensin II receptor blockers, nitrate preparations and cholesterol lowering drugs. The effect of ACE inhibitors is mainly based on their capability to improve the effect of bradykinin, which enhances the synthesis of nitric oxide in endothelium. Induced and/or enhanced nitric oxide production or added nitrates can balance insufficient internal nitric oxide production. The medicines and nitrates function as exogenic EDRF and dilate blood vessels, and in addition they are active as antithrombotic compound in damaged veins.

WO 02/34767 A1, Selwood et al., describes peptides which are fragments of vascular endothelial growth factor (VEGF), and useful for inhibiting angiogenesis. According to the invention, the peptides comprise three to eight amino acids, and a key feature of the amino acid sequence is the presence and arrangement of basic residues, in particular arginine (Arg) and/or lysine (Lys) residues. The document specifies several peptides, consisting of from six to sixteen amino acids. None of them include the sequences Isoleucin-Proline-Proline (IPP) or Valine-Proline-Proline (VPP). According to the publication, the peptides may be useful in diseases where angiogenesis plays a significant role in pathology. Such diseases may include diabetic retinopathy, age-related macular degeneration (ARDS), cancer, endometriosis, psoriasis and rheumatoid arthritis.

WO 99/45941, Sandberg et al., describes a composition used to enhance the softness, elasticity, or appearance of tissue. The composition is formulated from peptides that correspond to any one of 41 peptide fragments produced from thermolysing digestion of elastin. Preferably, the composition comprises a polypeptide having the formula R1-Valyl-Valyl-Prolyl-Glutamine-R2, wherein R1 is an amino portion of the peptide, and R2 is a carboxy portion of the peptide. The composition is preferably applied to human skin in a cosmetic formulation. According to the document, the composition may also be useful for treating hypertension, coronary heart disease, arteriosclerosis, angina, coronary thrombosis, chronic obstructive pulmonary disease, and restenosis post angioplasty.

WO 01/91700 A2 and U.S. Pat. No. 6,962,904 B1, both Mitts et al, are in part based on the same study as WO 99/45941, Sandberg et al. The documents describe compositions for enhancing the elasticity of tissue, and the compositions are formulated from peptides corresponding to sequences found in elastin, in particular the sequences -Valine-Valine-Proline- and -Valine-Valine-Proline-Asparagine-. Said compositions are useful for improving elastin production in tissues. According to the publications, the main utility is once again in cosmetics, but it is also mentioned that the compositions may be useful in treating e.g. hypertension, coronary heart disease, arteriosclerosis, angina, coronary thrombosis, chronic obstructive pulmonary disease and restenosis post-angioplasty.

The described elastin peptide fragments contain a large number of glycine and/or proline residues as compared to other amino acid sequences. The fragments do not include the sequences Isoleucin-Proline-Proline (IPP) and/or Valine-Proline-Proline (VPP).

Preparation of bioactive peptides by fermentation has been largely studied and described in the background art, one particular point of interest being milk-derived peptides. These have been shown to have for instance opioid receptor binding properties, ACE inhibiting activity and antimicrobial properties. Several studies have been made in relation to e.g. hypertension, but effects of the peptides on the endothelial functions have not been described. In particular, effects of the tripeptides Valine-Proline-Proline (VPP) and Isoleusine-Proline-Proline (IPP) and products containing the same on the endothelial functions have not been described in the background art.

The tripeptides VPP and IPP are known compounds, which have been described as ACE inhibitors having an antihypertensive effect. For instance in J Dairy Sci 78 (1995) 777-783, Nakamura et al. describe the use of a starter containing Lactobacillus helveticus and Saccharomyces cerevisiae for the preparation of two ACE inhibitors. The compounds were both identified as tripeptides, Val-Pro-Pro and Ile-Pro-Pro. Although the publication does not describe an in vivo antihypertensive effect of the tripeptides, it is mentioned to be the next subject of research.

U.S. Pat. No. 5,449,661, Nakamura et al., discloses the preparation of a peptide containing the tripeptide sequence Val-Pro-Pro and its use for lowering high blood pressure. The peptide is prepared by fermenting fat-free milk powder with the Lactobacillus helveticus strain JCM-1004, whereafter the peptide is purified chromatographically and freeze-dried.

In WO 99/16862, Yamamoto et al. describe the Lactobacillus helveticus strain CM4, FERM BP-6060, which is capable of producing a large amount of the tripeptides Val-Pro-Pro and/or Ile-Pro-Pro.

WO 01/32905, Valio, describes a product having antihypertensive properties and its preparation. The product is produced by fermenting a casein-containing starting material with a lactic acid bacterium, and performing nanofiltration on the obtained, peptide-containing fermentation product. The antihypertensive properties of the product are in part due to the tripeptides IPP and VPP contained therein. WO 03/070267, Valio, describes the use of IPP and VPP, as well as the product described in WO 01/32905, in the preparation of a product enhancing the availability of minerals. The product can be used e.g. for increasing bone formation, strengthening the skeleton system and for treating or prevention of osteoporosis.

Blood vessels have implications in diseases associated with viscoelasticity, including hypertension, arteriosclerosis, angina, angiogenesis, myocardial infarction, coronary thrombosis, restenosis post angioplasty, and chronic obstructive pulmonary disease. Cardiovascular diseases are amongst the most common diseases in the world, and they are on the top five list of life threatening diseases in many countries. Unfortunately, increasing living standards also increase the risk of said diseases, and hence they will play an even greater role in the future. In addition to conventional drugs, functional products are nowadays providing an attractive alternative to the consumers.

Functional products improving the elasticity of blood vessels and improving and normalizing endothelial function would hence be very welcome as part of a regular diet. Furthermore, administering peptides with beneficial arterial stiffness properties as a medical or pharmaceutical product is also worth considering.

### BRIEF DESCRIPTION OF THE INVENTION

It is thus an objective of the present invention to make available a product that as part of a regular diet, or as a medical and pharmaceutical product, improves or normalizes endothelial function and hence is capable of preventing, alleviating or curing disorders and diseases relating to endothelial dysfunction. Endothelial dysfunction has a remarkable role in the stiffness or flexibility of blood vessels, which in turn is important in many severe disorders including e.g. coronary heart disease, arteriosclerosis, angina, coronary thrombosis, chronic obstructive pulmonary disease and restenosis post-angioplasty. Hence, ability to enhance the elasticity of blood vessels is an especially important feature of the present invention.

The product to be used according to the invention consists of or comprises peptides improving endothelial function.

It is a further object of the present invention to make the product available either as such, as an agent improving endothelial function, or for use in the preparation of functional foodstuffs or drugs intended for consumption.

A yet further object of the present invention is to provide a method for prevention, alleviation or cure of endothelial dysfunction, as well as disorders and diseases relating thereto, by administering to an individual in need of such treatment peptides improving endothelial function or a product containing them in a sufficient amount to produce the desired effect.

A yet further object of the present invention is to provide a method for prevention, alleviation or cure of endothelial dysfunction, as well as disorders and diseases relating thereto, by administering to an individual a product that has a high content of casein-derived, small-molecular peptides and that has been prepared by fermenting a casein-containing starting material with Lactobacillus helveticus strain LBK-16H, DSM 13137, or Lactobacillus helveticus strain LB 1936, DSM 17754, and optionally removing partly or entirely casein and/or other milk proteins and/or lactose, in a sufficient amount to produce the desired effect. In a preferred embodiment, the fermented product is also subjected to nanofiltration.

### DETAILED DESCRIPTION OF THE INVENTION

According to the present invention it has been surprisingly found that the above objectives are achieved by using small-molecular peptides, in particular the tripeptides Ile-Pro-Pro (IPP), Val-Pro-Pro (VPP). Said peptides have now been proven capable of normalizing endothelial functions, improving the elasticity of blood vessels and combating arterial stiffness.

The peptides used in accordance with the present invention are bioactive peptides corresponding to those derived through hydrolysis of casein or casein-containing material, such as milk. Short-chain di-, tri- and tetrapeptides and their mixtures are considered suitable. In particular, the peptides are the tripeptides IPP and VPP, mixtures of peptides including IPP and VPP, and products and compositions including the same.

The preferred embodiment provides excellent possibilities to normalize endothelial dysfunction and endothelium dependent damages.

The invention thus relates to the use of casein-derived, small-molecular peptides in the preparation of a product improving endothelial function.

The invention further relates to the use of a product comprising casein-derived, small-molecular peptides in the preparation of a product improving endothelial function.

Preferably, the casein-derived, small-molecular peptides comprise di-, tri-, and tetrapeptides and their mixtures. Most preferably, the IPP and VPP contents are high.

The invention further relates to the use of a product having a high content of casein-derived, small-molecular peptides that has been prepared by fermenting casein-containing starting material with a lactic acid bacterium, and possibly by nanofiltration of the fermented peptide-containing product obtained, in the preparation of an end product improving endothelial function.

As a preferred embodiment, the invention relates to the use of a soured composition that contains casein-derived peptides, minerals and living lactic acid bacterium, in the preparation of a product improving endothelial dysfunction.

The invention also relates to a method for normalizing endothelial function and endothelium dependent damages by administering to an individual peptides normalizing endothelial function and endothelium dependent damages or a product containing them in a sufficient amount to produce the desired effect.

The individual is primarily human. The positive effects of the products used in accordance with the invention are naturally also beneficial to animals, especially pets and production animals. Examples of these include dogs, cats, rabbits, horses, cows, pigs, goats, sheep and poultry.

The tripeptides VPP and IPP can be prepared synthetically or by hydrolysis of material containing said sequences. In a preferred embodiment, the peptides are prepared from casein or casein-containing starting material by fermentation. Especially preferable is to use the method described in WO 01/32905, in which a product containing biologically active peptides is formed by fermentation, followed by concentration and nanofiltration. This preferred embodiment provides excellent possibilities to use starting materials of different types and to modify the composition of the end product as desired, as described in detail in WO 01/32905, which is incorporated herein by reference.

When biologically active peptides for use in accordance with the present invention are formed by fermentation, the starting material can be any product that contains the sequences of the desired biologically active peptides as part of its own peptide or protein sequence. Milk protein, especially casein, is preferably used as such or in the form of different preparations. Suitable starting materials also include various casein-containing milk products, such as skimmed milk or milk with varying fat contents as such or in the form of a corresponding milk powder and sour milk products, such as sour milk, buttermilk, yoghurt, curdled milk, unripened cheese, etc.

Fermentation can be carried out with any lactic acid bacterium that is capable of producing the desired peptides from the starting material. Suitable lactic acid bacteria can be found among species belonging to the Lactobacillus, Lactococcus, Leuconostoc, Streptococcus and Bifidobacterium genera, for instance. The most proteolytic of the lactic acid bacteria is Lactobacillus helveticus and it is thus considered especially suitable for this purpose. A preferable Lactobacillus helveticus strain is L. helveticus LBK-16H, DSM 13137, which is described in detail in patent publication WO 01/32836. Especially preferable is Lactobacillus helveticus LB 1936, DSM 17754, which is described in detail in patent application FI20065054.

The lactic acid bacteria can be used as pure cultures or mixed cultures, separately or together with conventionally used and commercially available souring agents. The lactic acid bacteria can also be used together with other micro-organisms.

The fermentation conditions are selected to meet the requirements of the used strain so as to form a sufficient amount of biologically active peptides to produce the desired effect. The selection of suitable conditions, such as temperature, pH and aeration, is part of the know-how of a person skilled in the. Conventionally fermentation is carried out at about 30 to 45° C. The fermentation is allowed to continue until the desired amount of biologically active peptides has been formed. Normally, this takes approximately 20 to 30 hours. A mixture of various peptides is formed during fermentation. When the fermentation continues long enough, mainly relatively small di- and tripeptides, such as Val-Pro-Pro (VPP) and Ile-Pro-Pro (IPP), are obtained. An incubation time of about 22 to 24 hours is preferred.

The cell suspension obtained can be used as such or the peptides can be separated and purified using conventional methods. Concentration, for instance by evaporation or drying the cell suspension partly or completely, such as spreading the suspension on a plate, drying and finally grinding it to a well-preserved dry powder, is a preferable treatment.

Further treatment of the cell suspension by nanofiltration has proven to be an excellent method that enables production of the peptides active in endothelial functions such as arterial stiffness, together with a desired mineral composition in the same product. By selection of the nanofiltration membrane type and process conditions, it is possible to influence the composition of the obtained product.

Nanofiltration is performed up to the desired dry content range, which may be about 20 to 40%, or to the approximate volume concentration ratio of about 5 to 20. By means of nanofiltration, the peptide content of the product increases. The composition of the end products naturally depends on the fermentation conditions used, the optional nanofiltration treatment and possible other pre-treatment and additional treatments.

In accordance with the present invention, the tripeptides IPP and VPP and products containing the same have surprisingly been shown to reduce arterial stiffness and thus improve endothelial dysfunctions. During a ten weeks intervention period the ambulatory arterial stiffness index (AASI) measuring the arterial stiffness improved significantly in the test group receiving a product comprising tripeptides IPP and VPP as compared to the control group; even in the first non-optimised test the index decreased about 9 percent in comparison to the control group. The average AASI at the end of the treatment period were 0.31±0.15 in the Lactobacillus helveticus group and 0.34±0.17 in the control group.

The products described above can be used as such to achieve the desired effect. The products can also be dried and used in the form of a powder or lyophilized preparation. The products can also preferably be used in the preparation of functional foodstuffs or other products. Applications as medical or pharmaceutical products are also possible.

The concept ‘foodstuff’ has a wide meaning in the present publication, covering all consumable products that can be in a solid, jellied or liquid form, and covering both ready-made products and products to which the biologically active peptides or a product containing them are added during consumption as an additive or part of the product. Foodstuffs can for instance be products of dairy industry and beverage industry. Typical products include milk products, such as yoghurt, curdled milk, curd, sour milk, sour whole milk, buttermilk, other sour milk products, unripened cheeses and ripened cheeses, filling of snack bars, etc. Another important group includes beverages, such as whey beverages, fruit beverages, and carbonated beverages.

According to the invention, the biologically active peptides or a product containing them are used in a sufficient amount to achieve the desired effect. When using the product obtained by the two-step method described above, the amount to use depends mainly on the concentration degree of the whey and is for instance 0.1 to 30%, preferably approximately 5 to 15% as calculated from the weight of the end product.

Biologically active peptides or a product containing them can be added to a food product during its preparation or to a finished food product. The food products in question thus contain the desired peptides, or a product containing them, in addition to other components contained in corresponding food products and fully correspond in taste and behaviour with these conventional products.

The invention will be described in detail by means of the following examples. These examples are provided only to illustrate of the invention and should not be considered to limit the scope of protection in any way. The examples set forth and present the beneficial, advantageous, and remedial effect of IPP and VPP as such or combined on arterial stiffness by enhancing endothelial function, as well as preferred end products for administering the bioactive peptides.

### EXAMPLE 1

### Effect of the Bioactive Product on Arterial Stiffness

Arterial stiffness can be measured by using ultrasound equipment or applanation tonometry, or by using ambulatory arterial stiffness index (AASI). AASI is defined as 1 minus the slope of diastolic or systolic pressure during 24-hour ambulatory monitoring, and it is a reliably indicator of arterial stiffness (Li Y, Wang J G, Dolan E, Gao P J, Guo H F, Nawrot T. Ambulatory arterial stiffness index derived from 24-hour ambulatory blood pressure monitoring. Hypertension 2006; 47(3):359-64).

In a randomised placebo-controlled parallel group study, 94 hypertensive patients not receiving any drug treatment were given either Lactobacillus helveticus LBK-16H fermented milk prepared according to Example 2 or a control product, for ten weeks after a four-week run-in period. Twenty-four hour ambulatory blood pressure measurement was performed at the beginning and at the end of intervention period. The average baseline systolic and diastolic blood pressure values were 132.6±9.9/83.0±8.0 mmHg in the Lactobacillus helveticus group and 130.3±9.6/80.2±7.0 mmHg in the control group.

At the end of the ten week intervention period systolic and diastolic blood pressure had decreased more in the Lactobacillus helveticus group compared to the control group. The treatment effect on SBP was −4.1 (95% Cl: −6.6 to −1.8, p<0.001). The treatment effect on DBP was −1.8 (95% Cl: −3.7 to −0.0, p=0.048).

AASI-index was determined by using the ambulatory blood pressure values and analysing these with the method of Li et al., ibid. The AASI index increases linearly with age in both men and women. Small values in AASI index are more favourable than high values and therefore, if the value decreases, the arterial stiffness index is getting better. The average baselines AASI were 0.36±0.15 in the Lactobacillus helveticus group and 0.36±0.17 in the control group. At the end of the treatment period average AASI were 0.31±0.14 in the Lactobacillus helveticus group and 0.34±0.15 in the control group.

During the ten week intervention period the AASI-index improved statistically significantly in the test groups (p=0.043), but not statistically in the control group (p=0.47) (Table 1).

TABLE 1
Change in AASI index after 10-week intervention period

| | | Sig. (2-tailed) | Mean Difference |
|---|---|---|---|
| Test group | Change in | 0.043 | −0.0427 |
| | AASI- | | |
| | index | | |
| Control | Change in | 0.474 | −0.0195 |
| group | AASI- | | |
| | index | | |

### EXAMPLE 2

### Preparation of Products Useful in the Present Invention

Lactobacillus helveticus strain LBK 16-H, DSM 13137, was grown in MRS broth at 37° C. for 24 hours and inoculated into reconstituted milk (10%) to form an inoculum. After two growth cycles, the inoculum (15%) was inoculated into a fermentor medium made up of 9 to 10% skimmed milk powder milk and sterilized at 110° for 10 minutes. Fermentation was performed at 37° C. for 22 to 24 hours under continuous strong agitation. The product (a) can be used as such, in a dry and/or ground form, or the desired peptides can be separated from it using methods known per se.

The process was repeated by using Lactobacillus helveticus LB 1936, DSM 17754, and rich milk or butter milk, respectively, instead of skimmed milk powder in order to produce corresponding products b and c.

### EXAMPLE 3

### Preparation of an End Product Useful in the Present Invention Peptide Concentrate

Sour milk containing peptides active in correcting endothelial dysfunction was prepared by adding about 1% of a peptide concentrate to a commercially available sour milk. The composition of the product is shown in Table 2, which for comparison also shows the composition of a commercially available sour milk product, AB sour milk, produced by Valio Ltd.

For the Evolus® peptide concentrate fermentation was carried out as described in example 2. The cell suspension obtained was separated by centrifugal clarifying. The thus pretreated whey was nanofiltrated through a Nanomax-50 membrane at 40° C. at a pressure of 30 bar. The whey was filtrated until the volume concentration ratio was 9.

TABLE 2
The composition of an Evolus product and a commercial sour
milk product (AB sour milk, Valio Ltd)

| Nutrition information/100 g | | | | | |
|---|---|---|---|---|---|
| (serving 200 g) | | Evolus | | AB sour milk | |
| energy | | 260 | kJ/61 kcal | 220 | kJ/53 kcal |
| protein | | 3.0 | g | 3.2 | g |
| carbohydrate | | 12 | g | 4.4 | g |
| of which lactose less than | | 1.0 | g* | | |
| dietary fibre | | 0 | g | 0 | g |
| fat | | 0.1 | g | 2.5 | g |
| calcium | | 150 | mg** | 120 | mg |
| potassium | | 233 | mg | 170 | mg |
| magnesium | | 13 | mg | 11 | mg |
| sodium | | 24 | mg | 39 | mg |
| Tripeptides | Ile-pro-pro | 1.1 | mg | 0 | |
| | Val-pro-pro | 1.3 | mg | 0 | |

*over 80% of the lactose has been enzymatically degraded
**25% of recommended daily intake

### EXAMPLE 4

### Preparation of an End Product Useful in the Present Invention Evolus Tehojuoma Fermented Milk Drink

For the Evolus® daily dose Tehojuoma fermented milk drink, fermentation was carried out as described in example 2. Further, 1% of Evolus® peptide concentrate prepared as described in example 3 was added to fermented milk. The product containing the daily requirement of the active ingredient is to be used as such. The nutrition information of the product is shown in Table 3. Further ingredients which fermented milk drink contains are pasteurized skimmed milk, milk protein concentrate, thickeners, sea algae calcium, acidity regulator, souring agents, flavours, vitamins (folic acid, B6, B12) and colour.

TABLE 3
The composition of Evolus ® Tehojuoma Fermented Milk Drink

| Nutrition information/100 g | EvolusTehojuoma | |
|---|---|---|
| (serving 100 ml) | mixed berry Drink | |
| energy | 260 | kJ/61 kcal |
| protein | 3.5 | g |
| carbohydrate | 11 | g |
| of which lactose less than | 1.0 | g* |
| dietary fibre | 0 | g |
| fat | 0.1 | g |
| calcium | 200 | mg** |
| potassium | 410 | mg |
| magnesium | 15.5 | mg |
| sodium | 38 | mg |
| folic acid | 66 | μg*** |
| vitamin B6 | 0.66 | μg*** |
| vitamin B12 | 0.33 | μg*** |

*over 80% of the lactose has been enzymatically degraded
**25% of recommended daily intake
***33% of recommended daily intake

### EXAMPLE 5

### Preparation of an End Product Useful in the Present Invention Soft Type of Cheese

Soft type cheese, such as fresh cheese, fresh cheese spread and cottage cheese were manufactured with conventional production methods. For example, Evolus® fresh cheese spread was produced by mixing quarg, cream or butter and other ingredients, heat-treated and packed. Evolus® peptide concentrate prepared as described in example 3 was added to quarg-fat mixture. The product containing the daily requirement of the active ingredient is to be used as such.

1. Use of lie-Pro-Pro and/or Val-Pro-Pro for the preparation of a product improving endothelial function.

2. The use according to claim 1, characterized by using a product comprising lie-Pro-Pro and/or Val-Pro-Pro for the preparation of a product improving endothelial function.

3. The use according to claim 2 of a product that has a high content of casein-derived, small-molecular peptides and has been prepared by fermenting a casein-containing starting material with a lactic acid bacterium for the preparation of a product improving endothelial function.

4. The use according to claim 3 of a product that has a high content of casein-derived, small-molecular peptides and has been prepared by fermenting a casein-containing starting material with Lactobacillus helveticus strain LBK-16H, DSM 13137, or Lactobacillus helveticus strain LB 1936, DSM 17754, optionally removing partly or entirely the casein and/or other milk proteins and/or lactose, and nanofiltering the resulting peptide-containing fermented product, for the preparation of a product improving endothelial function.

5. The use according to claim 2 of a soured composition containing the casein-derived, small-molecular peptide Ile-Pro-Pro and/or Val-Pro-Pro, and living lactic acid bacterium for the preparation of a product improving endothelial function.

6. The use of a product according to claim 3, characterized in that the casein-derived, small-molecular peptides comprise a mixture of short-chain peptides.

7. Use according to claim 1 for the preparation of a medical product for prevention or treatment of disorders and diseases relating to endothelial dysfunction.

8. Use according to claim 1, characterized in that the product enhances the elasticity of blood vessels.

9. Use according to claim 1, characterized in that the end product is an edible product.

10. Use according to claim 9, characterized in that the end product is a product of dairy industry, beverage industry, processed food industry, processed meat industry, baking industry or confectionery industry.

11. Use according to claim 10, characterized in that the end product is a fermented milk product.

12. Use according to claim 10, characterized in that the end product is a beverage, preferably a whey beverage, a fruit beverage or a beer.

13. Use according to claim 9, characterized in that the end product is a product of pharmaceutical industry.

14. A method for normalizing endothelial dysfunction, comprising administering to an individual a peptide selected from the group consisting of lie-Pro-Pro and Val-Pro-Pro and mixtures thereof, or a product containing said peptide(s), in a sufficient amount to achieve the desired effect.

15. A method for prevention or treatment of endothelial dysfunction, and disorders and diseases relating thereto, by administering to an individual a peptide selected from the group consisting of lie-Pro-Pro and Val-Pro-Pro and mixtures thereof, or a product containing said peptide(s), in a sufficient amount to achieve the desired effect.
